(19) United States
(12) Patent Application Publication
(10) Pub. No.: US 2006/0154986 A1
(43) Pub. Date: Jul. 13, 2006

(54) A TRIACYLGLYCERIDE TO DELIVER HEALTH PROMOTING FATTY ACIDS TO THE UPPER GASTROINTESTINAL TRACT AND SKIN

(75) Inventors: John Westcott Finley, Lansdale, PA (US); Igor A. Mezine, Malvern, PA (US)

Correspondence Address: THADDIUS J. CARVIS, 102 NORTH KING STREET, LEESBURG, VA, 20176, US

(73) Assignee: Thaddius J. Carvis, Leesburg, VA (US)

(21) Appl. No.: 11/275,495

(22) Filed: Jan. 10, 2006

Related U.S. Application Data
(60) Provisional application No. 60/642,931, filed on Jan. 11, 2005.

(51) Int. Cl. A61K 31/22

(52) U.S. Cl. 514/547

(57) ABSTRACT

Improved triglyceride compositions, bearing both omega-3-usaturated fatty acids and short chain fatty acids, are prepared according to the formulae: 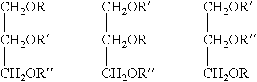 wherein, each R, independently, is a fatty acid residue, either saturated or unsaturated, having between 2 and 40 carbons; each R′, independently, is a long chain omega-3-unsaturated fatty acid residue having between 16 and 40 carbons, preferably 18 to 22 carbons; and each R″, independently, is a short chain acid residue having 2 to 5 carbons, preferably 2 to 4 carbons, most preferably 2 to 3 carbons. Also described is a regimen for treating a medical problem associated with less than healthy stomach tissue that is responsive to treatment with a triglyceride mixture as defined above, in amounts and at intervals effective to therapeutically improve the health of the stomach tissue. In another aspect the compositions are used in a regime for treating skin to increase skin cell turnover, to provide nourishment to epithelial cells and reduce inflammation. The regime comprises topically administering to a mammal a triglyceride mixture as defined above, in amounts and at intervals effective for these purposes.

### RELATED APPLICATION

This application claims priority to U.S. Provisional Patent Application No. 60/642,931 filed Jan. 11, 2005, the disclosure of which is incorporated herein by reference.

### BACKGROUND OF THE INVENTION

The invention relates to improvements foods by providing new triacylglycerol compositions to deliver health promoting fatty acids to the upper gastrointestinal tract and to promote healthy looking skin.

The pharmaceutical industry has provided antacid medications for mild stomach distress. These remedies are very helpful to some people, but many taking them have already suffered damage to stomach tissue. Currently, the antacid medications deal with the acid causing the symptoms of burning, without taking care of the stomach tissue itself.

Other drugs, like esomeprazole, have been developed for the treatment of erosive esophagitis associated with Gastro-Esophageal Reflux Disease (GERD), which is virtually universal among age groups. In most people it remains physiologic. When the frequency or duration of GERD becomes severe enough to induce symptoms or histologic changes of chronic inflammation, GERD becomes pathologic. GERD has been shown to play an important role in acute and chronic inflammatory disorders of the airway. In addition, there is a growing body of evidence that GERD plays a significant role in the development of a number of diseases and symptom complexes, including cancer of the larynx, laryngeal and tracheal stenosis, reflux laryngitis, globus, chronic cough, otitis media, and sinusitis. See, for example, Levinson, Sidman and Brown, Sinusitis in Children—Diagnosis and Treatment, The Medical Journal of Allina, Vol. 5, No. 1, Winter 1995. Esomeprazole, is a proton pump inhibitor (PPI) that suppresses the final step in gastric acid production. It acts by suppressing gastric acid secretion by specific inhibition of the H+/K+-ATPase in the gastric parietal cells. While effective, it does nothing for the damage tissue.

Another cause of stomach tissue damage are NSAIDs, which despite impressive therapeutic benefits, place individuals using them at risk for several serious complications. The major complication is gastrointestinal toxicity, which is evident even at low doses of aspirin used for prevention of myocardial infarction. In fact NSAID toxicity is considered to be the number one cause of drug-induced complications. NSAID-related ulcer complications lead to up to 400,000 hospitalizations and 16,500 deaths yearly in the United States. It would be desirable to find a simple way to restore damaged mucosal tissue or maintain its health to a greater extent in the face of gastric insults occasioned by NSAIDs or the like.

The mammalian skin is also in need of care and nourishment. The skin is made of three layers: epidermis, dermis and fat. The outer layer is the epidermis, which contains sheets of epithelial cells called keratinocytes. These keratinocytes are produced at the junction between the epidermis and the second layer of skin, the dermis. The epidermis is supported from below by the dermis. The epidermis contains many layers of closely packed cells. The cells nearest the skin's surface are flat and filled with a tough substance called keratin. The epidermis contains no blood vessels—these are all in the dermis and deeper layers. The epidermis is thick in some parts (one millimeter on the palms and soles) and thin in others (just 0.1 millimeter over the eyelids). Dead cells are shed from the surface of the epidermis as very fine scale, and are replaced by other cells which pass from the deepest (basal) layers to the surface layers over a period of about four weeks. The dead cells on the surface take the form of flattened, overlapping plates, closely packed together. This layer is known as the stratum corneum and is characterized by its flexibility, being more or less waterproof and has a dry surface that it is inhospitable to micro-organisms. The dermis is made up of connective tissue, which contains a mixture of cells that give strength and elasticity to the skin. This layer also contains blood vessels, hair follicles and roots, nerve endings, and sweat and lymph vessels and glands. The elements of the dermis all carry messages or fluids to and from the epidermis so it can grow, respond to the outside world and react to what goes on inside the body. Underneath the dermis is a layer of fat which acts as an important source of energy and water for the dermis. It also provides protection against physical injury and the cold.

Certain fatty constituents associated with mammalian nutrition have been associated with aspects of health. Omega-3 fatty acids have a variety of beneficial properties in the diet and have been widely suggested as important. It is known, for example, that omega-3 fatty acids are therapeutically effective for reducing inflammation in the stomach wall. Short chain fatty acids have been less discussed in the literature, but are known as a preferred energy substrate for the stomach mucosa. In U.S. Pat. No. 5,780,451, DeMichele, et al., describe an enteral nutritional product for a person having ulcerative colitis. It contains in combination an oil blend which contains eicosapentaenoic acid (20:5n3) and/or docosahexaenoic acid (22:6n3), and a source of indigestible carbohydrate which is metabolized to short chain fatty acids by microorganisms present in the human colon. There is, however, no recognition that an effective therapeutic composition for treating skin and/or mucosal tissues can be formed wholly of fatty materials, which can be used effectively in food compositions.

The provision of fat in the diet is important for a variety of reasons from taste and texture, to nutrition and flavor. Fats have been associated in the literature with full range of positive and negative effects on health. While research is ongoing and no clear answers with regard to their full effect on health and nutrition can be drawn at this time, it is clear that they form an essential part of the mammalian diet and are sought after in many forms.

Fats are predominantly triglycerides, i.e., triacylglycrides. When fats are consumed, they are digested by a complex series of mechanisms that depend on the fat composition and other factors. Because the fatty acid constituents become available for their biological effects upon digestion, it would be desirable to provide a group of fats useful as foods, which would also enable a simple and convenient means for assisting in maintenance of digestive tract mucosa. As aids to moisturizing skin, fats would also be a useful aid to use in promoting healthy skin.

There is a need for improvements in providing remedies for stomach tissue damage and it would be desirable to do so with food approaches to aid the health of mammalian stomach tissue.

There is also a need to treat mammalian skin to make it more healthy, give it a more healthy appearance, and/or reduce inflammation.

### SUMMARY OF THE INVENTION

It is an object of the invention to provide a composition, which is suitable as a food and is therapeutically effective for reducing inflammation in the stomach wall.

It is another object of the invention to provide a composition, which is suitable as a food and provides a preferred energy substrate for the mucosa.

It is yet a more specific object of the invention to provide a composition which is a suitable food ingredient and is therapeutically effective for reducing inflammation in the stomach wall and provides a preferred energy substrate for the mucosa, thereby promoting healthy stomach tissue when consumed.

It is yet a more specific object of the invention to provide an improved regimen for treatment of a patient having less than fully healthy stomach tissue.

It is an object of a preferred aspect of the invention to provide a composition and treatment regimen for treatment of a mammalian patient to promote healthy stomach tissue, particularly for humans and domestic animals.

It is another object of the invention to provide a composition to treat mammalian skin to make it more healthy, give it a more healthy appearance, and/or reduce inflammation.

It is yet another object of the invention to provide a regimen to treat mammalian skin to make it more healthy, give it a more healthy appearance, and/or reduce inflammation.

These and other objects are accomplished by the invention, which provides compositions suitable for food and cosmetic uses and treatment regimens employing them.

In one aspect the invention provides a mixture of one or more triglyceride compounds containing omega-3 fatty acids and short chain fatty acids, according to the formulae: 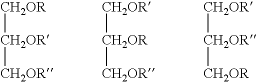
wherein,
each R, independently, is a fatty acid residue, either saturated or unsaturated, having between 2 and 40 carbons;
each R′, independently, is a long chain omega-3-unsaturated fatty acid residue having between 16 and 40 carbons, preferably 18 to 22 carbons; and
each R″, independently, is a short chain acid residue having 2 to 5 carbons, preferably 2 to 4 carbons, most preferably 2 to 3 carbons.

In another of its aspects the invention provides a regimen for treating a medical problem associated with less than healthy stomach tissue that is responsive to treatment with a triglyceride mixture as defined above, in amounts and at intervals effective to therapeutically improve the health of the stomach tissue.

In another of its aspects the invention provides a regimen for treating mammalian skin to make it more healthy, give it a more healthy appearance, and/or reduce inflammation.

Preferred aspects of the invention will be described below.

### DESCRIPTION OF THE INVENTION

The invention is intended to provide therapeutic aid and relief to mammalian subjects, especially humans, but also other treatable mammals including dogs, cats, horses, goats, sheep, cattle, and other domesticated animals and pets suffering problems associated with less than healthy stomach tissue. Throughout this discussion, the reference to stomach tissue is meant to include like mucosal tissue throughout the digestive tract.

A triacylglycride (TAG) has been made that will deliver fatty acids to the upper gastrointestinal tract that promote cellular health and reduce inflammation. The TAG contains a mixture of one or more triglyceride compounds containing omega-3 fatty acids and short chain fatty acids, according to the formulae: 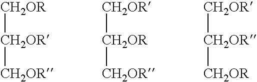
wherein,
each R, independently, is a fatty acid residue, either saturated or unsaturated, having between 2 and 40 carbons;
each R′, independently, is a long chain omega-3-unsaturated fatty acid residue having between 16 and 40 carbons, preferably 18 to 22 carbons; and
each R″, independently, is a short chain acid residue having 2 to 5 carbons, preferably 2 to 4 carbons, most preferably 2 to 3 carbons.

The R fatty acid residues in the above formulae can be any suitable fatty acid, including one of the omega-3 fatty acid residues, a short chain fatty acid or any other fatty acid selected for its effect on the physical properties of the fatty acid mixture or other beneficial effect. The R′ omega-3 fatty acid residues in this composition are therapeutically effective for reducing inflammation in the stomach wall. The short chain R″ fatty acid residues in this composition are a preferred energy substrate for the mucosa.

The composition is preferably prepared as a TAG mixture of compounds meeting this formulation. The mixture is composed primarily of triglycerides containing at least one R″ short chain fatty acid (acetic, propionic or butyric) and at least one R′ long chain fatty omega-3 fatty acid (linolenic, stearidonic, arachadonic, eicosapentaenoic, docosapentaenoic or docosahexaenoic). Other R fatty acid residues will preferably be present to enable provision of the properties normally associated with food fats. In some embodiments the R fatty acid residues will be omega-3 fatty acid residues. In others, the R fatty acids will comprise short chain fatty acids. In yet others the R fatty acid residues will comprise one or more saturated or unsaturated long chain fatty acids or medium chain fatty acids. As used herein, the term “acid residue” refers to an acyl group comprising a carbon chain and a carbonyl group. Acylation of a glycerol hydroxyl by acid ROH results in the attachment of R chain acid residue to the glycerol backbone by means of an ester linkage (—O—(CO)—).

One especially preferred embodiment comprises triglyceride mixtures having an acyl carbon number (ACN) of 20 to 40 and the R and R′ residues are predominantly omega-3 acid residues. In another embodiment, the triglyceride mixtures have an acyl carbon number of 18 to 30 and the long residues are predominantly short chain acid residues.

Depending upon the preparative procedure (more fully discussed below), triglyceride mixtures of this invention may also contain R groups comprising residues of unsaturated long chain fatty acids. They may also contain R groups comprising residues of saturated long chain fatty acids. In yet other embodiments, the R groups can comprise residues of medium chain fatty acids.

In the practice of this invention, medium chain residues can be incorporated into triglycerides bearing short and omega-three fatty acid residues in amounts sufficient to achieve desirable physical properties in the fat, such as a lower melting profile or a more liquid range. For example, the insertion of from about 1 to 15% of medium residues can provide useful results. In some cases up to 30% can be useful. Preferred medium chain groups are derived from predominantly, e.g., at least about 50%, a mixture of caprylic and capric acids.

Short chain R″ acid residues may be straight or branched and may be derived from any synthetic or natural organic acid including, but not limited to acetic (ethanoic), propionic (propanoic), butyric (butanoic), and the like acids. As used herein, chemical names include isomeric variations; for example, “butyric acid” includes normal-butyric acid (butanoic) and iso-butyric (2-methylpropanoic) acid, and so forth. Preferred acids are acetic, butyric, mixtures of acetic and butyric, mixtures of acetic and propionic, and mixtures of acetic, propionic, and butyric.

Medium chain R fatty acid residues are derived from any synthetic or natural organic medium chain fatty acid as that term is understood in the art, including, but not limited to caproic (hexanoic), caprylic (octanoic), pelargonic (nonanoic), capric (decanoic), lauric (dodecanoic) and the like acids. Preferred medium chain fatty acids contain predominantly, e.g., at least about 50%, preferably at least about 75% caprylic acid or capric acid, or mixtures of these acids.

The long saturated R fatty acid residues are derived from fatty acid residues having 16 to 40, preferably 16 to 22, carbons. These long chain saturated residues may be derived from any synthetic or natural, straight or branched saturated organic acid including, but not limited to, palmitic (hexadecanoic), stearic (octadecanoic), arachidic (eicosanoic), behenic (docosanoic), and the like acids.

Unsaturated long R groups may also be present in the mixtures. These are derived from unsaturated acids such as, but not limited to, palmitoleic (9-hexadecenoic), oleic (cis-9-octadecenoic), elaidic (trans-9-octadecenoic), vaccenic (trans-11-octadecenoic), linoleic (cis, cis-9,12-octadecedienoic), linolenic (9,12,15-octadecatrinoic and 6,9,12-octadecatrienoic), eleostearic (9,11,13-octadecatrienoic), arachidonic (5,8,11,14-eicosatetraenoic), and the like acids. Some sources rich in omega-3 fatty acids will also contain other unsaturated long chain fatty acids.

Any or all of the fatty acid residues useful in the invention may be derived from mixtures of acids, such as, for example, those derived from specific fractions of unhydrogenated, partially hydrogenated or fully hydrogenated dairy butterfat, coconut, palm kernel and the like oils and fats. For example, butterfat has been fractionated, yielding a fraction enriched with triglycerides having 2 residues of at least 16 carbons and 1 residue with 2 to 8 carbons (U.S. Pat. No. 4,479,976 to Lansbergen and Kemps, and U.S. Pat. No. 4,504,503 to Biernoth and Merk). Various long chain fatty acid residues can be derived from mixtures of fatty acids obtained from natural oils such as soybean, safflower, sunflower, sesame, peanut, corn, olive, rice bran, mustard seed, cottonseed, poppyseed, rapeseed, marine, meadowfoam and the like oils; fats such as babassu nut oil, palm oil, tallow, lard, and shea butter; or plant waxes such as jojoba. Fat mixtures and/or fractions, crystallized fats, interesterified fats and mixtures of these may also be employed.

Mixtures of R groups may preferably derived from oils and fats that are hydrogenated, most preferably fully hydrogenated, to provide oxidative stability and more solid content to the oils where they are to be used in foods requiring these properties. Fully hydrogenated fats typically have an Iodine Value of 5 or less, and, in some cases, less than 2. Hydrogenated fats having at least about 70%, preferably at least about 75%, stearic acid residues such as, for example, hydrogenated peanut oil, hydrogenated olive oil, hydrogenated soybean oil, hydrogenated sesame oil, and hydrogenated corn oil are especially desirable. Some embodiments employ fatty acid residues derived from hydrogenated fats having at least about 90% stearic acid residues, such as hydrogenated sunflower oil, hydrogenated safflower oil and hydrogenated canola. Embodiments having fatty acid residues exhibiting a high in behenic acid content can be derived from hydrogenated rapeseed, hydrogenated fish oil, and hydrogenated meadowfoam.

Other embodiments employ fatty acid residues derived from a mixture of hydrogenated oils having pendant groups exhibiting greater chain length variety, such as, for example, from a mixture of hydrogenated canola or soybean oil and rapeseed oil, hydrogenated canola or soybean oil and cottonseed oil, and the like. Because there is evidence that trans unsaturated fatty acids, and palmitic acid, lauric acid and myristic acid may increase plasma cholesterol concentrations (Bonanome, A., and Grundy, S. M., New Eng. Jour. Med. 318: 1244-1248 (1988)), preferred feedstocks are low in palmitic acid, lauric acids and partially hydrogenated fatty acid contents. It is an advantage of the invention, however, that beneficial attributes can be obtained with TAGs of the invention including these materials in minor amount, preferably less than 10% total by weight.

Some triglyceride mixtures of this invention contain about 15 to about 55 mole % short fatty acid residues, about 15 to about 60 mole % medium fatty acid residues, and about 20 to about 80 mole % long fatty acid residues, both of the omega-3 and other species. The molar ratio of short to the sum of omega-3 residues of some preferred embodiments ranges between about 0.5 to 2.0, and will collectively comprise at least 50% of the fatty acid species.

When the TAG mixtures are consumed, the short chain fatty acids are rapidly hydrolyzed in the stomach by gastric lipase and absorbed as energy for the mucosa. These fatty acids result in improved cell health and increased cell turnover in the same way that the short chain fatty acids in the colon promote colonic health. After hydrolysis the omega-fatty acids present on mono-and di-glycerides which facilitates their uptake and utility as anti-inflammatory fatty acids for the stomach and intestinal mucosa.

This invention also provides a regimen for treating a medical problem associated with less than healthy stomach tissue that is responsive to regimen for treating a medical problem associated with less than healthy stomach tissue that is responsive to the novel fats of the invention in amounts and at intervals effective to therapeutically improve the health of the stomach tissue. The effective level of the TAG mixtures will be determined by suitable assay, such as by testing for C-reactive protein as a marker by the method described for example by J. Wilkins, Clinical Chemistry 44: 1358-1361, 1998, to show a positive response of significant therapeutic effect. Other markers and tests for them are also available. The size of the dose is not critical, and there is no upper limit known for such. The novel fats of the invention will preferably be employed in capsule form or in food products including any of those containing a fat component, including spreads such as margarine, salad dressings, crackers, dips, snack chips, and the like. The amounts per dose will typically contain at least 1 gram of the novel triglycerides, and the doses can contain more, e.g., from 2 to 25 grams. In some cases, doses or daily dosage amounts of from 25 to 100 grams can be employed. Higher and lower doses can be effective when properly administered and monitored.

This invention also provides a regimen for treating skin of mammals to make it healthier by promoting a more rapid cell turnover and provide direct nourishment to epithelial cells. When these compounds are directly applied to the skin of mammalian patient having skin conditions or diseases treatable by short chain triglycerides, the subject compounds are partially hydrolyzed to provide energy to the epithelial cells and humectancy to the skin. For this application, acetic and propionic acids are preferred to butyric acid, due to the odor, but there may be applications that favor the use of butyric. For this application, the R groups will preferably predominate as saturated fatty acids. The C:18 omega-3 from flax may offer some similar benefits with slightly better oxidative stability.

In this embodiment related to skin care, the short chain fatty acids are hydrolyzed off of the compounds, and the resulting monoglycerides are absorbed through the skin. Prior to adsorption they act as humectants on the surface of the skin. The result of the treatment of the skin with the subject compounds is that the cell turnover is greater, and reduce inflammation and/or provide energy sources to the epithelial cells. The net effect is a younger looking skin, which can be objectively evaluated. A preferred regimen for use is to apply the compounds of the invention to the skin liberally at least once daily, preferably after bathing the area to be treated. A more preferred regimen will entail topical application twice daily. The amount per treatment will be determined based on the surface area in need of treatment. The compounds of the invention should be applied in amounts sufficient to fully cover the skin area being treated and can be rubbed in to promote absorption if that is practical.

Synthesis should be conducted with recognition of the sensitivity of the omega-3 fatty acids and the need to preserve their natural chemical structure for maximum therapeutic effect. With this in mind, they can be made by known esterification techniques, including transesterification and random interesterification, among others. Some preparative procedures for triglycerides bearing short and long substituents have been published. Interesterification of a shortening base stock with triacetin yielded an improved plastic product (though all the Examples employed partially, not fully, hydrogenated base stocks; see U.S. Pat. No. 2,614,937 to Baur and Lange). Acetylated monoglycerides have been discussed above. An acetylated monoglyceride prepared from lard, cottonseed oil or partially hydrogenated vegetable oil has been disclosed as useful in lowering cholesterol (U.S. Pat. No. 4,272,548 to Gatzen, et al.). Triglycerides bearing palmitic and butyric residues were synthesized for study as pancreatic lipase and human milk bile lipase substrates (in Clement, G., et al., Biochem. Biophys. Res. Commun. 8: 238-242 (1962) and Wang, C. S., et al., cited previously, respectively). See, also, U.S. Pat. No. 5,258,197 and U.S. Pat. No. 6,713,447.

The following examples are presented to further illustrate and explain the invention and are not to be taken as limiting in any regard. Unless otherwise indicated, all parts and percentages are by weight.

### EXAMPLE 1

Five ml of solution of sodium methoxide (0.5M in MeOH) was evaporated on a rotovap and flask was charged with 15 g (50 mM) of tributyrine (Fluka). After stirring for 10 min, fish oil triglycerides (35/25 TG Fish oil; Ocean Nutrition Canada, Ltd.) was added drop wise (m=14 g), the flask was flashed with nitrogen, and the reaction mixture stirred overnight at room temperature. The Reaction mixture was diluted with hexane (100 ml) and washed with 50 ml of 50% EtOH containing 0.1% phosphoric acid, 50 ml of 50% EtOH, 50 ml of brine. The hexane layer was dried over anhydrous CaCl₂ and evaporated on a rotovap to yield 25.5 g (˜90%) as clear yellow-brownish oil.

The above description is for the purpose of teaching the person of ordinary skill in the art how to practice the invention. It is not intended to detail all of those obvious modifications and variations, which will become apparent to the skilled worker upon reading the description. It is intended, however, that all such obvious modifications and variations be included within the scope of the invention which is defined by the following claims. The claims are meant to cover the claimed components and steps in any sequence which is effective to meet the objectives there intended, unless the context specifically indicates the contrary.

1. Improved triglyceride compositions, bearing both omega-3-usaturated fatty acids and short chain fatty acids, according to any of the formulae: 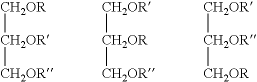
wherein,
each R, independently, is a fatty acid residue, either saturated or unsaturated, having between 2 and 40 carbons;
each R′, independently, is a long chain omega-3-unsaturated fatty acid residue having between 16 and 40 carbons, preferably 18 to 22 carbons; and
each R″, independently, is a short chain acid residue having 2 to 5 carbons, preferably 2 to 4 carbons, most preferably 2 to 3 carbons.

2. Improved triglyceride compositions according to claim 1 wherein at least some of the R groups comprise long chain omega-3-unsaturated fatty acid residues having between 16 and 40 carbons.

3. Improved triglyceride compositions according to claim 2 wherein at least some of the R groups comprise long chain omega-3-unsaturated fatty acid residues having between 18 to 22 carbons

4. Improved triglyceride compositions according to claim 1 wherein at least some of the R groups comprise short chain acid residues having 2 to 5 carbons.

5. Improved triglyceride compositions according to claim 4 wherein at least some of the R groups comprise short chain acid residues having 2 to 3 carbons

6. Improved triglyceride compositions according to claim 1 wherein at least some of the R groups comprise long chain saturated fatty acid residues having between 16 and 40 carbons.

7. Improved triglyceride compositions according to claim 1 wherein at least some of the R groups comprise long chain saturated fatty acid residues having between 18 to 22 carbons.

8. Improved triglyceride compositions according to claim 1 wherein at least some of the R groups comprise medium chain fatty acid residues having between 6 and 10 carbons.

9. A regimen for treating a medical problem associated with less than healthy stomach tissue that is responsive to treatment with a triglyceride mixture as defined above, in amounts and at intervals effective to therapeutically improve the health of the stomach tissue.

10. A regimen that improves the outcome of patients with a condition or disease connected with fibromylagia, Chron's disease, irritable bowel syndrome and other inflammatory conditions in the gastrointestinal tract, comprising orally administering to a patient so affected a triglyceride mixture as defined above, in amounts and at intervals effective to therapeutically improve the noted condition or disease.

11. A regime wherein the lipids in claim 1 are combined with NSAID's to help prevent the gastrointestinal side effects of the drugs comprising orally administering to a patient so affected a triglyceride mixture as defined above with an NSAID, in amounts and at intervals effective to therapeutically improve the noted condition or disease.

12. A regimen for treating skin to increase skin cell turnover, and reduce inflammation and/or to provide nourishment to epithelial cells, comprising topically administering to a mammalian skin a triglyceride mixture as defined in claim 1, in amounts and at intervals effective for these purposes.
